(19) United States
(12) Patent Application Publication
(10) Pub. No.: US 2023/0266312 A1
(43) Pub. Date: Aug. 24, 2023

(54) DIAGNOSTIC IMMUNOASSAY STRIPS, DEVICES AND METHODS TO DETECT AND VALIDATE BIOLOGICAL SAMPLE

(71) Applicant: Varun AKUR VENKATESAN, Koramangala (IN)

(72) Inventors: Varun AKUR VENKATESAN, Koramangala (IN); Siddharth PATTNAIK, Koramangala (IN)

(21) Appl. No.: 18/012,914

(22) PCT Filed: Jun. 25, 2021
(86) PCT No.: PCT/IN2021/050622

§ 371 Date: Dec. 24, 2022

(30) Foreign Application Priority Data
Jun. 25, 2020 (IN) 202011020802

(51) Int. Cl. G01N 33/543

(52) U.S. Cl. CPC G01N 33/54387

(57) ABSTRACT

An immunoassay device that authenticates a biological sample while performing an assay for an analyte or analytes of interest is provided. The device includes a sample receiving zone to receive the biological sample; a validation zone placed before or after the sample receiving zone to validate the biological sample; a conjugate zone having labels conjugated with primary antibodies or reagents specific to a plurality of characteristic markers of the biological sample and the analytes of interest; and a reaction zone having secondary antibodies or antigens or reagents specific to the primary antibodies or reagents that bind with the plurality of characteristic markers of the biological sample and analytes of interest.

### FIELD OF INVENTION

The present embodiment relates to diagnostic immunoassay devices and strips and methods, and more particularly to the immunoassay strips and devices such as lateral or vertical flow assays strips or immunoassay microfluidic devices and related methods/devices to authenticate and/or detect presence of a biological sample, and authenticate the volume.

### SUMMARY OF THE PRIOR ART

In view of the foregoing, devices, strips, assays and methods for authenticating a biological sample is provided.

In an aspect, an immunoassay device that authenticates a biological sample while performing an assay for an analyte or analytes of interest is provided. The device includes a sample receiving zone to receive the biological sample; a validation zone placed before or after the sample receiving zone to validate the biological sample;
a conjugate zone having labels conjugated with primary antibodies or reagents specific to a plurality of characteristic markers of the biological sample and the analytes of interest; and a reaction zone having secondary antibodies or antigens or reagents specific to the primary antibodies or reagents that bind with the plurality of characteristic markers of the biological sample and analytes of interest. The device is a lateral flow immunoassay strip or vertical flow immunoassay strip or microfluidic immunoassay device. The conjugate zone has labelled primary antibodies/chemicals/reagents specific to a plurality of constituents of a buffer. The reaction zone has labelled secondary antibodies/chemicals/reagents specific to the labelled primary antibodies conjugated with the plurality of constituents of the buffer. The biological sample is a bodily fluid. The biological sample is blood or urine or sweat or semen or serum. The validation zone juts out of the immunoassay device for visualisation.

In another aspect, a method for performing authentication of a biological sample in an immunoassay device while performing an assay for a plurality of analytes of interest is provided. The method includes adding the biological sample to the immunoassay device; allowing the biological sample to pass through a validation zone on to a sample zone or allowing the biological sample to pass to the validation zone via the sample zone or allowing the biological sample to split with one part going into the validation zone and other part on to a reaction zone; reacting the biological sample with chemicals/reagents/antibodies to cause a coloured, fluorescent or electrochemical reaction; and imaging the coloured or fluorescent reaction to authenticate the biological sample. The method further includes allowing the biological sample to pass through a conjugate zone. The biological sample is a bodily fluid. The method further includes authenticating a buffer by conjugating a plurality of constituents of buffer with labels conjugated with primary antibodies/reagents to obtain conjugated buffer; and reacting the conjugated buffer with secondary antibodies/antigens/reagents with the primary antibodies conjugated with labels to cause a coloured, fluorescent or electrochemical reaction. The method further includes authenticating sequence and timing of addition of buffer by capturing a video image of the device and analysing the video image for the sequence and time lapse between addition of the biological sample and the buffer. The method further includes electing a criterion for validation of the biological sample based on the detection of plurality of biological markers.

In yet another aspect, a lateral flow immunoassay strip or device that authenticates a biological sample while performing an assay for an analyte or analytes of interest is provided. The method includes a validation pad to receive the biological sample; a sample pad placed in proximity to the validation pad to receive the biological sample through the validation pad; a conjugate pad that has a plurality of antibodies against a plurality of characteristic markers in the biological sample and the analytes of interest; and a reaction pad that has a plurality of test and control zones with immobilised reagents/antibodies that bind with the primary antibodies conjugated with the plurality of characterising markers and the analytes of interest. The validation pad juts out of the strip or device; and is placed below or above the sample pad. The lateral flow immunoassay strip or device includes a detection membrane of a fixed thickness corresponding to a threshold quantity of the biological sample to be received.

In still another aspect, a vertical flow immunoassay strip or device that authenticates a biological sample while performing an assay for an analyte or analytes of interest is provided. The device includes a sample introduction zone; a sample pad to receive the biological sample through the validation pad; a validation pad to receive the biological sample from the sample pad; a conjugate pad that has a plurality of labelled primary antibodies/chemicals/antigens against a plurality of characteristic markers in the biological sample and the analytes of interest; and a reaction pad that has a plurality of test and control lines having immobilised secondary reagents/antibodies that bind with the primary antibodies conjugated with the plurality of characterising markers and the analytes of interest.

In still another aspect, a microfluidics immunoassay device that authenticates a biological sample while performing an assay for an analyte or analytes of interest is provided. The microfluidics device includes a sample input channel to allow passage of the biological sample having a number of capillaries of different diameters in which the biological sample is separated in different fractions based on capillary diameters; and a sample authentication zone having antibodies/chemicals/reagents specific to characteristic markers of the biological sample to cause a coloured or fluorescent reactions; and an analyte detection zone having antibodies/chemicals/reagents specific to an analyte of interest to cause coloured or fluorescent reactions.

In still another aspect, a method of detecting and validating a biological sample in a lateral flow immunoassay device while performing an assay for an analyte of interest is provided. The method includes receiving the biological sample on a detection membrane of a lateral flow assay strip, wherein the detection membrane is placed on a sample pad; imaging the detection membrane; and analysing the detection membrane for a coloured front that validates presence of the biological sample.

In still another aspect, a computer-implemented method for validating a threshold quantity of a biological sample that is received to perform an assay on an immunoassay strip or device comprising detecting a coloured front created at a validation zone of a thickness corresponding to the threshold quantity of the biological sample, wherein the coloured front is created when the thickness of the detection membrane corresponds to the threshold biological sample volume.

In still another aspect, a method for authenticating or approving an immunoassay for an analyte of interest in a biological sample is provided. The method includes authenticating the biological sample as true sample by obtaining a coloured reaction of a plurality of characteristic markers of the biological sample; and validating volume of the biological sample when the volume of the biological sample meets a threshold volume of the biological sample required for the immunoassay of the analyte of interest.

### BRIEF DESCRIPTION OF DRAWINGS

Other objects, features, and advantages of the embodiment will be apparent from the following description when read with reference to the accompanying drawings. In the drawings, wherein like reference numerals denote corresponding parts throughout the several views:

FIG. 1 illustrates a block diagram of an immunoassay device or strip that also authenticates validity of a biological sample, according to an embodiment herein;

FIGS. 2A, 2B and 2C illustrate a diagnostic lateral flow immunoassay strip with an internal control to authenticate a biological sample, according to an embodiment herein;

FIGS. 3A and 3B illustrates different blood sample volumes as received on test strips of varying thickness 3.5 mm and 1.77 mm respectively, according to an embodiment herein;

FIG. 4 illustrates a method of authenticating a biological sample in an immunoassay, according to an embodiment herein;

FIG. 5 illustrates a diagnostic vertical flow immuassay strip with an internal control to authenticate a biological sample, according to an embodiment herein; and

FIG. 6 illustrate an immunoassay microfluidics device with an internal control to authenticate a biological sample, according to an embodiment herein.

### DESCRIPTION OF THE PREFERRED EMBODIMENT

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The terms “pad”, “membrane” or “zone” or “channels” are used interchangeably across the present disclosure and are indicated to mean the same unless otherwise stated. They usually means the spots or area on a strip or a microfluidic device where the biological sample interacts with several reagents/antibodies/antigens involved of an immunoassay.

The “strip” is referred to as diagnostic assay strip or lateral flow assay strip or a vertical flow assay strip or an immunoassay strip unless otherwise stated. The strip is used interchangeably with device.

The “authentication” refers to identification of presence of components that validate the sample is a valid biological sample or correct for application or refers to verification that volume applied is correct or refers to validation or verification of the time of application as correct.

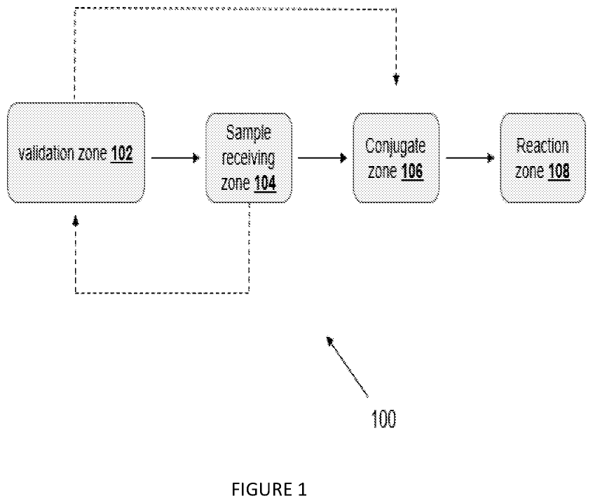

Referring now to the drawings, and more particularly to FIG. 1 through FIG. 6, where similar reference characters denote corresponding features consistently throughout the figures, there are shown several embodiment.

As mentioned, there remains a need to develop test strips, devices, readers and methods to authenticate or validate a biological sample while conducting an assay.

The embodiment herein provides an immunoassay strip or device that allows authentication of a valid biological sample as well as the authentication of the volume of the biological sample required for performing an immunoassay through FIG. 1. Figure illustrates a block diagram of an immunoassay device or strip 100 that allows authentication of a valid biological sample and its volume. The device 100 includes a validation zone 102, a sample receiving zone 104, a conjugate zone 106, and a reaction zone 108. The biological sample may be any bodily fluid such as sweat, blood, urine, serum, or semen. The biological sample may or may not include an analyte of interest such as a hormone or a metabolite. The biological sample includes characteristic markers.

In an embodiment, the validation zone 102 may be placed before or after the sample receiving zone 104. The alternative embodiment is shown by way of dotted-lines in FIG. 1. The validation zone 102 validates volume of the biological sample for a particular analyte assay. In an embodiment, the validation zone also validates sequence of addition of buffer for e.g., usually buffer is to be added after the biological sample has been added; accordingly, such a validation is confirmed by video imaging the validation zone 102.

The sample receiving zone 104 receives the biological sample having (or not having) an analyte and its characteristic markers. In an embodiment, the sample travels to the conjugate zone 106. In another embodiment, the sample travels to the conjugate zone 106 via the validation zone 102. The conjugate zone 106 contains primary antibodies/reagents/antigents that are tagged with labels such as gold, latex or fluorophore for binding with analyte, if present as well as with the characteristic markers of the biological sample. Thus, the conjugate zone 106 of the device 100 according to an embodiment herein also includes antibodies or reagents tagged with labels for binding with characteristic markers of the biological sample that in the downstream, thus, allows authentication or validation of the biological sample. From the conjugate zone 106, the conjugated biological sample flows to the reaction zone 108 where secondary antibodies/reagents/antigens bind with the primary antibodies/reagents/antigens leading to a coloured or fluorescent or electrochemical signal or reaction, thus proving presence or absence of the analyte while authenticating the biological sample. Thus, at the same time, the tagged primary antibodies conjugated with characteristic markers of the biological sample react with the secondary antibodies specific to the primary antibodies thus leading to a coloured or fluorescent or electrochemical signal or reaction validating or authenticating a biological sample. The reaction zone 108 consists of a number of test lines and control lines such that there is at least one test line, having an immobilised secondary antibody/antigen/reagent specific to a labelled primary antibody/antigen/chemical conjugated with an analyte of interest, and at least one test line having an immobilised secondary antibody/antigen/reagent specific to a labelled primary antibody/antigen/chemical conjugated with a characteristic biomarker of the biological sample. In an embodiment, it may be required that in order to authenticate a biological sample, presence of at least or minimum two characteristic markers is established. Accordingly, there may be two control lines each with an immobilised secondary antibody/antigen/reagent specific to a labelled primary antibody/antigen/chemical conjugated with two or more characteristic biomarkers of the biological sample.

Further, the immunoassay strip or device 100 also allows for authentication of a buffer in such assays in which the addition of the biological sample is followed by the addition of a buffer. In order to authenticate a buffer, a number of tagged reagents or antibodies specific to one or more constituents of the buffer are included in conjugate zone 106. In a preferred embodiment, the buffer is allowed to split and travel to a separate buffer conjugation zone (not shown in figure) having tagged reagents or antibodies specific to any constituent of the buffer. As the contents of buffer are now conjugated, either via travelling through the conjugate zone 106 or via the buffer conjugation zone, the buffer then travels to the reaction zone 108 that has further test lines having immobilised secondary antibodies/reagents/chemicals specific to the primary antibodies/reagents/chemicals conjugated to the constituents of buffer thus giving rise to coloured, or fluorescent or electrochemical reaction specific to buffer.

The device 100 further includes a sink to receive overflowing or excess biological sample and buffer. The reaction zone 108 is then scanned or read by an imaging device to analyse coloured fronts specific to analyte [whose presence or absence is to be detected in the biological sample], biological sample and buffer, if needed. In an embodiment, the device is inserted into an optical device having a transparent optic defining an optical volume. The transparent optic is adapted to admit into the optical volume a light emitted by the light source for illuminating the reaction zone 108 and the transparent optic is adapted to admit the light having interacted with the reaction zone 108, into the optical volume and turn the light inside the optical volume such that the light is internally reflected within the optical volume and exit the optical volume to be incident onto the camera of the handheld device. Alternatively, the device having the strip 100 of the present embodiment can be inserted into a stand-alone or specialised optical readers or devices meant to detect presence or absence of an analyte in a bodily fluid or biological sample.

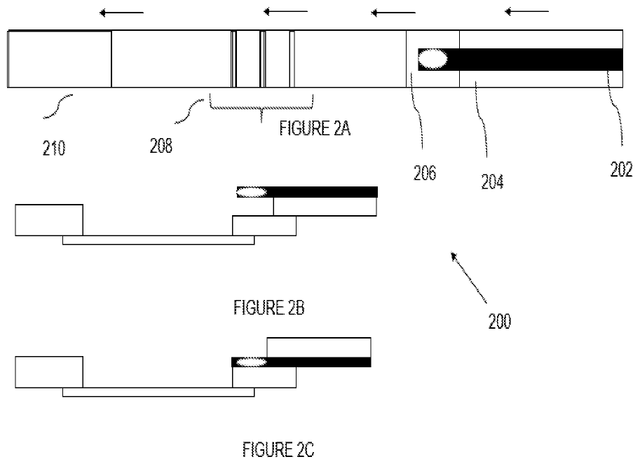

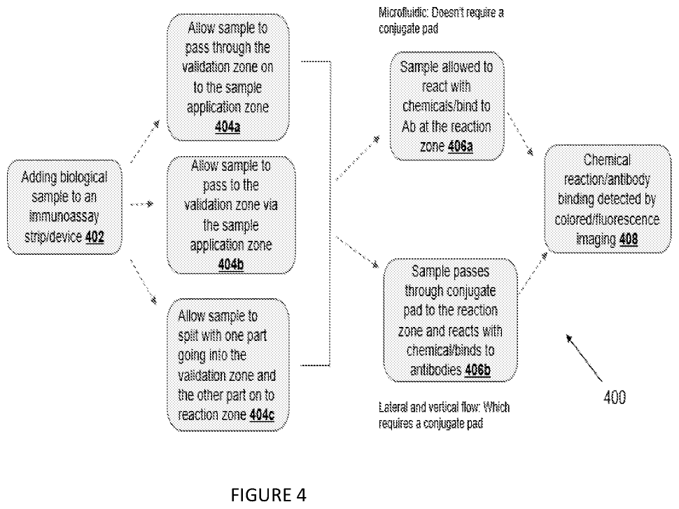

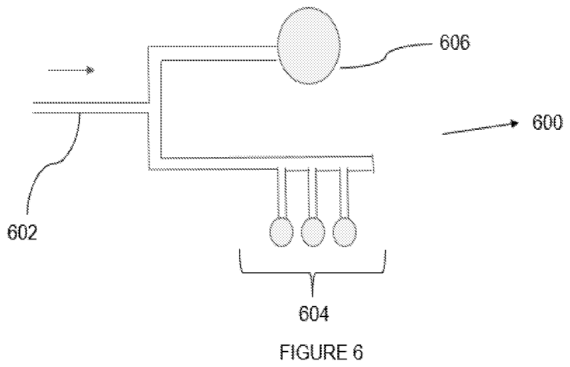

The immunoassay device 100 may be implemented as a lateral flow immunoassay device or strip (as depicted in FIGS. 2A, 2B and 2C), as a vertical flow immunoassay device or strip (as depicted in FIG. 4) or as a microfluidic immunoassay device or strip (as depicted in FIG. 6.

The lateral flow immunoassay strip 200 as depicted in FIGS. 2A, 2B and 2C includes a validation membrane 202 to detect an amount of biological sample added or received on the strip 200. FIGS. 2B and 2C depict two side view of two embodiments of the immunoassay device 200. In FIG. 2A, the validation membrane 202 is placed above a sample receiving pad or membrane 204; whereas, in FIG. 2B the validation membrane 202 is placed below a sample receiving pad or membrane 204. The strip 200 further includes the sample receiving pad 204, a conjugate pad 206, a reaction membrane or pad or zone 208 and an absorbent pad 210. In the lateral flow assay strip 200, the biological sample flows in lateral direction as the numerous pads are based on capillary beds. The validation membrane 202 partially protrudes from the strip 200 for visualization by an imaging device.

The flow of the biological sample e.g., blood sample, to be analysed is shown by way of arrows in FIG. 2, in which the biological sample flows laterally from the sample pad 204 after it is received by the sample pad 204 from the validation pad 202. In an embodiment, the biological sample is blood or any fraction of blood such as platelets or plasma that can be placed in the sample receiving pad or zone 204. In another embodiment, the biological sample is urine or any bodily fluid. In an embodiment, the validation pad's 202 position is below or on top of the sample receiving pad 204 and overlaps with the conjugate pad 206 to prevent interference of Red Blood Cells (RBCs) with the assay. The absorbent pad 210 is essentially a sink pad to receive/absorb extra amount of the biological sample that may be received on the strip 200.

In an embodiment, the strip 200 further is required to allow receipt of a buffer for assays where a buffer solution may be required to be added after introducing the biological sample. The buffer travels through the validation pad 202 and the conjugate pad 206.

The sample receiving pad 204 receives the biological sample having (or not having) an analyte and its characteristic markers. In an embodiment, the sample travels to the conjugate pad 206. In another embodiment, the sample travels to the conjugate pad 206 via the validation pad 202. The conjugate pad 206 contains primary antibodies/reagents/antigents that are tagged with labels such as gold, latex or fluorophore for binding with analyte, if present as well as with the characteristic markers of the biological sample. Thus, the conjugate pad 206 of the device 200 according to an embodiment herein also includes antibodies or reagents tagged with labels for binding with characteristic markers of the biological sample that in the downstream, thus, allows authentication or validation of the biological sample. From the conjugate pad 206, the conjugated biological sample flows to the reaction pad 208 where secondary antibodies/reagents/antigens bind with the primary antibodies/reagents/antigens leading to a coloured or fluorescent or electrochemical signal or reaction, thus proving presence or absence of the analyte while authenticating the biological sample. Thus, at the same time, the tagged primary antibodies conjugated with characteristic markers of the biological sample react with the secondary antibodies specific to the primary antibodies thus leading to a coloured or fluorescent or electrochemical signal or reaction validating or authenticating a biological sample. The reaction pad 208 consists of a number of test lines and control lines such that there is at least one test line, having an immobilised secondary antibody/antigen/reagent specific to a labelled primary antibody/antigen/chemical conjugated with an analyte of interest, and at least one test line having an immobilised secondary antibody/antigen/reagent specific to a labelled primary antibody/antigen/chemical conjugated with a characteristic biomarker of the biological sample. In an embodiment, it may be required that in order to authenticate a biological sample, presence of at least or minimum two characteristic markers is established. Accordingly, there may be two control lines each with an immobilised secondary antibody/antigen/reagent specific to a labelled primary antibody/antigen/chemical conjugated with two or more characteristic biomarkers of the biological sample.

In an aspect, a test strip for determining whether an assay meets minimum biological sample volume requirement is provided. The test strip includes a validation pad 202 with a fixed thickness that is correlated to the threshold quantity or volume of the biological sample required to conduct a particular assay of an analyte of interest. Should the quantity of biological sample received on the validation membrane 202 not correspond to the thickness such that the biological sample quantity is lower than the threshold, the coloured front on the validation membrane 202 is not visible when image analysis using handheld camera or any optoelectronic device or strip readers is performed, and the strip 200 is thus rejected.

In an embodiment, the strip 200 is adapted to be read by an optical device such that the reaction pad 208's test and control lines as well as the validation pad 202 are read using smartphone's or any handheld device's camera and light source to detect presence or absence of, as well as quantify, any constituent of any bodily fluid or the biological sample including the markers of the biological sample.

In a preferred embodiment, the validation membrane 202 allows serum [having analytes of interest, and the markers of serum] to travel towards the reaction membrane 208 while retaining Red Blood Cells (RBCs) in the blood sample to prevent interference by RBCs in the analysis. The RBCs are made to travel or flow towards absorbent pad 210 upon addition of buffer. In a preferred embodiment, thickness of the detection membrane 202 is varied depending upon quantity of the biological sample that is required to detect an analyte of interest. The sensitivity of the biological sample detection may thus be tweaked. Different assays may require different minimum amount of biological sample to provide an optimal reading or analysis. The test strip 200 thus may have a thickness of the validation membrane 202 that ensures the assay is performed only when the volume of the biological assay is proportional to the thickness of the detection membrane or meets a minimum or threshold criteria as determined by the thickness of the validation membrane 202.

In an embodiment, the strip 200 is used for assays for detecting and quantifying hormones, antibodies, metabolites and/or any biomolecule of interest such as glucose, bilirubin etc. In a preferred embodiment, the strip 200 is used for validating/authenticating any biological sample such as bodily fluids, blood, urine, semen etc. based on their characterizing biomarkers. The characterizing or characteristic biomarkers for blood include serum albumin, serum creatinine, fibrinogen, lipids, cholesterol. The characterizing biomarkers for urine includes Albumin, creatinine, urea, ammonia.

FIG. 4 illustrates a method 400 for detection of and quantifying of blood sample in diagnostic immunoassay devices or strips for authenticating presence and volume of a biological sample. The method includes adding a biological sample on or in a sample receiving zone in step 402. This is followed by allowing sample to pass through the validation zone 102 on to the sample receiving zone 104. In another embodiment, the biological sample is allowed to travel to the validation zone 102 via the sample receiving zone 104, in alternative step 404b. Alternatively, in step 404c, the biological sample is allowed to split and travels to the validation zone and reaction zone. In the step 406a, applicable to microfluidic devices, the biological sample then travels to the reaction zone, directly from the sample receiving zone, and the reaction zone has antibodies/antigens/reagents specific to the analyte of interest and characteristic markers of the biological sample. In another embodiment, applicable to lateral or vertical flow assay strips/device, in step 406a, the biological sample passes through the conjugate zone 106 to travel to the reaction zone 108 and reacts with chemical/binds to antibodies as explained in the foregoing.

The method further includes imaging the reaction zone and/or validation zone in step 408 via a camera [such as a smartphone camera] and analysing for a coloured front. The amount of biological sample is then analysed or computed and subsequently validated with respect to the visibility of the coloured front. Thus, an image processing of the detection zone is conducted to determine, quantify and authenticate the blood sample. In an embodiment, the method includes imaging the detection zone and/or reaction zone via a camera [such as a smartphone camera] and analysing for a red front to analyse or compute and subsequently validate the biological sample as blood with respect to the visibility of the coloured front. A red coloured front authenticates a blood sample.

In an aspect, an assay that requires a threshold quantity of a biological sample to be received on the strip is provided such that the device 100 is rejected when the volume of the biological sample is less than the threshold quantity. Whether a blood sample volume qualifies for a particular assay is determined by fixing thickness of detection membrane 202. If a particular assay requires a biological sample such as blood sample to be 10 μl, 5 μl, 2 μl, 1.5 μl, or 1 μl, the detection membrane 202 is made of thickness that is correlated to the volume to be assessed.

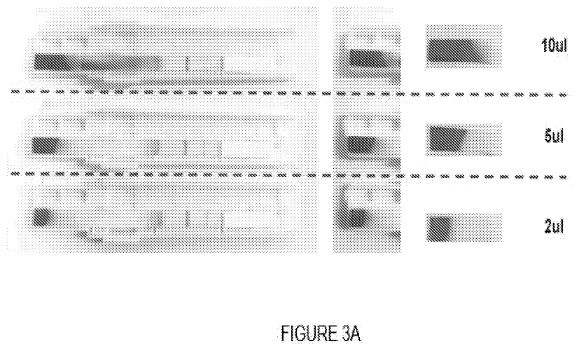

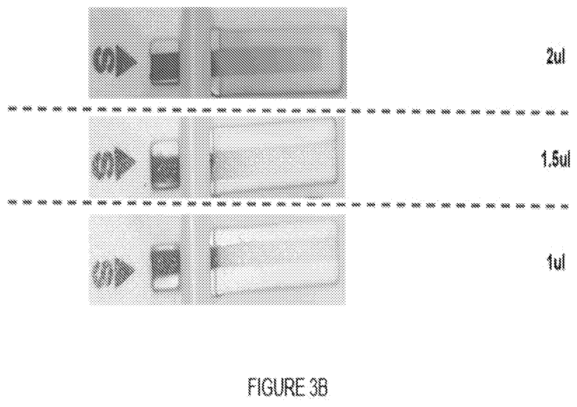

FIGS. 3A and 3B illustrate relation between blood sample volume and thickness of the detection strip. FIG. 2A depicts the detection membrane 202 of thickness correlated to 10 μl blood sample volume (in an embodiment, the thickness is 3.5 mm for 10 μl blood sample) and illustrates blood samples of volume 5 μl and 2 μl fail to make red front that is visible under an imaging sensor, thereby leading to failure of test. FIG. 2B depicts the detection membrane 202 of thickness correlated to 2 μl blood sample volume (in an embodiment, the thickness is 1.7 mm or 10 μl blood sample) and illustrates blood samples of volume 1.5 μl and 1 μl fail to make red front that is visible under an imaging sensor, thereby leading to failure of test.

In an aspect, a test strip for determining whether an assay meets minimum biological sample volume requirement is provided. The test strip includes a detection membrane 202, on the test strip 200's sample pad 204, with a fixed thickness that is correlated to the threshold quantity or volume of the biological sample required to conduct a particular assay. Should the quantity of biological sample received on the detection membrane 202 not correspond to the thickness such that the biological sample quantity is lower than the threshold, the coloured front on the detection membrane is not visible when image analysis using handheld camera or any optoelectronic device or strip readers is performed, and the strip 200 is thus rejected.

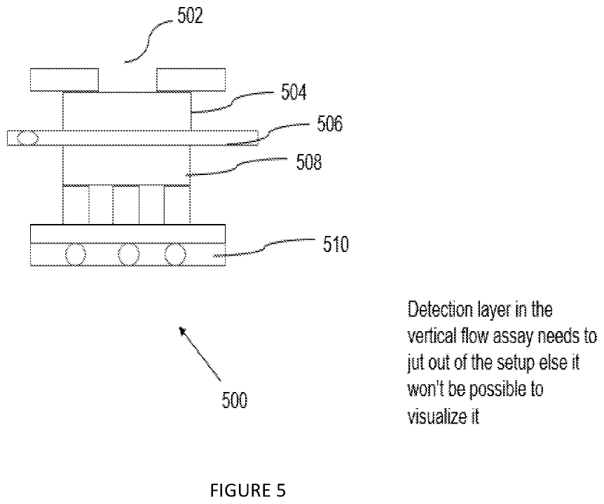

FIG. 5 illustrates a diagnostic vertical flow immunoassay strip 500 with an internal control to authenticate a biological sample, according to an embodiment herein. The diagnostic vertical flow immunoassay strip 500 includes a sample introduction zone 502, a sample receiving pad 504, a validation pad 506, a conjugate pad 508, and a reaction pad 510. The sample is introduced from the sample introduction zone 502, from there the biological sample travels to the sample pad 504 under gravity or external pressure. The validation pad 506 is placed below the sample pad 504 and juts out of the vertical flow strip/device 500 for visualisation under imaging device. The biological sample travels to the conjugate pad 508 includes primary antibodies/chemicals/reagents/antigens to bind with an analyte of interest as well as with one or more characteristic markers of the biological sample. Subsequently, the conjugated biological sample travels to the reaction pad 510. The reaction pad 510 includes test lines having immobilised secondary antibodies specific to the labelled primary antibodies conjugated with the analytes of interest and characteristic markers. The reaction pad 508 and the jutting portion of the detection pad 506 is visualised under an imaging device for visualisation of coloured/fluorescent reaction. In an embodiment, the validation pad 506, the conjugate pad 508 and the reaction pad 510 receive a buffer for assays where an analyte of interest requires buffer to be added subsequent to addition of the biological sample. The buffer is thus validated and authenticated using methods explained in the foregoing description.

FIG. 6 illustrate a microfluidics device 600 with a detection zone to authenticate a biological sample, according to an embodiment herein. The microfluidics device 600 includes a sample input channel 602 to allow passage of a biological sample. In an embodiment, the sample input channel includes a number of capillaries of different diameters in which the biological sample is separated in different fractions based on capillary diameters, e.g., serum flows in a thinner capillary. The sample then travels to analyte detection zones 604 and a biological sample authentication zone 602. The analyte detection zone 604 includes antibodies and/or chemicals specific to an analyte to cause reactions. The sample authentication zone 602 includes antibodies and/or chemical reagents specific to characteristic markers of the biological sample to cause a coloured or fluorescent reactions that may be read by reader devices having camera such as a smartphone to read the intensity of the reaction. In an embodiment, the device 600 further includes a buffer validation channel for the assays where a buffer solution may be required to be added after introducing the biological sample.

The strips or devices 100 as represented by 200, 500 or 600 may be prepared with different detection membrane thickness for different assays depending on requirement of minimum quantity or threshold quantity of biological sample required to perform a particular assay.

In an embodiment, a diagnostic assay, employing any of the devices 100, 200, 500 and 600 may require use of a buffer to be added after the biological sample introduced into the sample receiving zone or channels. The buffer authentication thus may also include to verify whether the buffer was added in right sequence and within a specific time frame. The strips or microfluidics devices are subject to an imaging module to record a video such that time lapse and order of introduction of the biological sample and buffer is recorded. Generally, the buffer is added after the biological sample; however, if the sequence is reversed the test is invalidated based on the video. Further, the method includes computing or determining time lapse, from the video, between addition of the biological sample and the buffer such that if the time lapse between addition of buffer after the introduction of the biological sample is more than a threshold time, the test is invalidated. The threshold time may vary as per diagnostic assays.

In an embodiment, a computer-implemented method for determining whether an assay meets minimum biological sample volume requirement for performing an assay is provided. The assay may be performed on lateral or vertical flow assay strips or microfluidics devices. The method includes detecting a coloured front created at the detection spot or channel. In strips-based assays, when the thickness of the detection membrane corresponds to the threshold biological sample volume, the volume is authenticated.

In an embodiment, the test strips or microfluidic devices include authentication zone or channels that validate a biological sample or buffer using electrochemical analysis such as by measuring impedance.

In an aspect, a method for approving an immunoassay for an analyte of interest in a biological sample is provided. The method includes authenticating the biological sample as true sample by obtaining a coloured reaction of a plurality of characteristic markers of the biological sample; and validating volume of the biological sample when the volume of the biological sample meets a threshold volume of the biological sample required for the immunoassay of the analyte of interest. The imaging device upon noticing coloured reactions specific to the characteristic markers of the biological sample at the relevant test lines in the reaction zone confirms the biological sample to be authentic sample. The imaging device upon noticing a coloured front at the validation zone authenticates the volume based on colour intensity at the validation zone. Once, the biological sample meets both the conditions, the assay is approved.

As will be readily apparent to those skilled in the art, the present embodiment may easily be produced in other specific forms without departing from its essential characteristics. The present embodiment are, therefore, to be considered as merely illustrative and not restrictive, the scope being indicated by the claims rather than the foregoing description, and all changes which come within therefore intended to be embraced therein.

I/we claim:

1. An immunoassay device (100) that authenticates a biological sample while performing an assay for an analyte or analytes of interest, comprising:
a sample receiving zone (104) to receive the biological sample;
a validation zone (102) placed before or after the sample receiving zone (104) to validate the biological sample;
a conjugate zone (106) having labels conjugated with primary antibodies or reagents specific to a plurality of characteristic markers of the biological sample and the analytes of interest; and
a reaction zone (108) having secondary antibodies or antigens or reagents specific to the primary antibodies or reagents that bind with the plurality of characteristic markers of the biological sample and analytes of interest.

2. The immunoassay device (100) as claimed in claim 1, wherein the device (100) is a lateral flow immunoassay strip (200) or a vertical flow immunoassay strip (500) or microfluidic immunoassay device (600).

3. The immunoassay device (100) as claimed in claim 1, wherein the conjugate zone (106) has labelled primary antibodies/chemicals/reagents specific to a plurality of constituents of a buffer.

4. The immunoassay device (100) as claimed in claim 1, wherein the reaction zone (108) has labelled secondary antibodies/chemicals/reagents specific to the labelled primary antibodies conjugated with the plurality of constituents of the buffer.

5. The immunoassay device (100) as claimed in claim 1, wherein the biological sample is blood or urine or sweat or semen or serum.

6. The immunoassay device (100) as claimed in claim 1, wherein the validation zone (102) juts out of the immunoassay device (100) for visualisation.

7. A method (400) for performing authentication of a biological sample in an immunoassay device while performing an assay for a plurality of analytes of interest, comprises:
adding (402) the biological sample to the immunoassay device;
allowing (404a) the biological sample to pass through a validation zone (102) on to a sample zone (104) or allowing (404b) the biological sample to pass to the validation zone (102) via the sample zone (104) or allowing (404c) the biological sample to split with one part going into the validation zone (102) and other part on to a reaction zone (108);
reacting (406a) the biological sample with chemicals/reagents/antibodies to cause a coloured, fluorescent or electrochemical reaction; and
imaging (408) the coloured or fluorescent reaction to authenticate the biological sample.

8. The method as claimed in claim 7 further comprises allowing (406b) the biological sample to pass through a conjugate zone (106).

9. The method as claimed in claim 7, wherein the biological sample is a bodily fluid.

10. The method as claimed in claim 7 further comprising authenticating a buffer by conjugating a plurality of constituents of buffer with labels conjugated with primary antibodies/reagents to obtain conjugated buffer; and reacting the conjugated buffer with secondary antibodies/antigens/reagents with the primary antibodies conjugated with labels to cause a coloured, fluorescent or electrochemical reaction.

11. The method as claimed in claim 10 further comprising authenticating sequence and timing of addition of buffer by capturing a video image of the device (100, 200, 500 and 600) and analysing the video image for the sequence and time lapse between addition of the biological sample and the buffer.

12. The method as claimed in claim 10 further comprising electing a criterion for validation of the biological sample based on the detection of plurality of biological markers.

13. A lateral flow immunoassay strip or device (200) that authenticates a biological sample while performing an assay for an analyte or analytes of interest, comprising:
a validation pad (202) to receive the biological sample;
a sample pad (204) placed in proximity to the validation pad (202) to receive the biological sample through the validation pad (202);
a conjugate pad (206) that has a plurality of antibodies against a plurality of characteristic markers in the biological sample and the analytes of interest; and
a reaction pad (208) that has a plurality of test and control zones with immobilised reagents/antibodies that bind with the primary antibodies conjugated with the plurality of characterising markers and the analytes of interest.

14. The lateral flow immunoassay strip or device (200) as claimed in claim 13 wherein the validation pad (202) juts out of the strip or device (200); and is placed below or above the sample pad (204).

15. The lateral flow immunoassay strip or device (200) as claimed in claim 13 for validating a threshold volume of a biological sample while performing an assay for the analyte of interest comprising a detection membrane (202) of a fixed thickness corresponding to a threshold quantity of the biological sample to be received.

16. A vertical flow immunoassay strip or device (500) that authenticates a biological sample while performing an assay for an analyte or analytes of interest, comprising:
a sample introduction zone (502);
a sample pad (504) to receive the biological sample through the validation pad (502);
a validation pad (506) to receive the biological sample from the sample pad (504);
a conjugate pad (508) that has a plurality of labelled primary antibodies/chemicals/antigens against a plurality of characteristic markers in the biological sample and the analytes of interest; and
a reaction pad (208) that has a plurality of test and control lines having immobilised secondary reagents/antibodies that bind with the primary antibodies conjugated with the plurality of characterising markers and the analytes of interest.

17. A microfluidics immunoassay device (600) that authenticates a biological sample while performing an assay for an analyte or analytes of interest, comprising:
a sample input channel (602) to allow passage of the biological sample having a number of capillaries of different diameters in which the biological sample is separated in different fractions based on capillary diameters;
a sample authentication zone (606) having antibodies/chemicals/reagents specific to characteristic markers of the biological sample to cause a coloured or fluorescent reactions; and
an analyte detection zone (604) having antibodies/chemicals/reagents specific to an analyte of interest to cause coloured or fluorescent reactions.

18. A method of detecting and validating a biological sample in a lateral flow immunoassay device (200) while performing an assay for an analyte of interest, comprising:
receiving the biological sample on a detection membrane (202) of a lateral flow assay strip (200), wherein the detection membrane (202) is placed on a sample pad (204);
imaging the detection membrane (202); and
analysing the detection membrane (202) for a coloured front that validates presence of the biological sample.

19. A computer-implemented method for validating a threshold quantity of a biological sample that is received to perform an assay on an immunoassay strip or device (100) comprising detecting a coloured front created at a validation zone (102) of a thickness corresponding to the threshold quantity of the biological sample, wherein the coloured front is created when the thickness of the detection membrane corresponds to the threshold biological sample volume.

20. A method for approving an immunoassay for an analyte of interest in a biological sample comprising:
authenticating the biological sample as true sample by obtaining a coloured reaction of a plurality of characteristic markers of the biological sample; and
validating volume of the biological sample when the volume of the biological sample meets a threshold volume of the biological sample required for the immunoassay of the analyte of interest.
